(19) United States
(12) Patent Application Publication
(10) Pub. No.: US 2020/0179449 A1
(43) Pub. Date: Jun. 11, 2020

(54) METHODS FOR GENERATING POLYCLONAL REGULATORY T CELLS

(71) Applicant: The Trustees of Columbia University in the City of New York, New York, NY (US)

(72) Inventors: Megan Sykes, New York, NY (US); Adam Griesemer, New York, NY (US)

(21) Appl. No.: 16/615,529

(22) PCT Filed: Jun. 12, 2018
(86) PCT No.: PCT/US18/37073

§ 371 Date: Nov. 21, 2019

Related U.S. Application Data
(60) Provisional application No. 62/518,384, filed on Jun. 12, 2017.

(51) Int. Cl. A61K 35/17; C12N 5/0783; C12N 13/00; A61P 37/06

(52) U.S. Cl. CPC A61K 35/17; C12N 5/0637; C12N 13/00; C12N 2501/2302; C12N 2502/1107; C12N 2502/1114; A61P 37/06

(57) ABSTRACT

The present disclosure provides for methods to produce B cell-expanded Tregs that are more stable and more effective than donor-specific Tregs for suppressing polyclonally activated T cells. In certain embodiments, the present polyclonal Tregs can react to many donors, not just a specific donor.

### CROSS-REFERENCE TO RELATED APPLICATIONS

The present application claims priority to U.S. Provisional Patent Application Ser. No. 62/581,384 filed Jun. 12, 2017, which is incorporated herein by reference in its entirety.

### STATEMENT OF GOVERNMENT SUPPORT

This invention was made with government support under grant number R01 OD017949 awarded by the National Institutes of Health. The government has certain rights in this invention.

### FIELD OF THE INVENTION

The present disclosure provides for methods to produce B cell-expanded Tregs that are more stable and more effective than donor-specific Tregs for suppressing polyclonally activated T cells. In certain embodiments, the present polyclonal Tregs can react to many donors, not a specific donor.

### BACKGROUND

Regulatory T (Treg) cells are immunosuppressive and generally suppress or downregulate induction and proliferation of effector T cells. Bettelli et al., 2006, Reciprocal developmental pathways for the generation of pathogenic effector TH17 and regulatory T cells, Nature, 441 (7090): 235-238. Treg cells (Tregs) prevent immune responses to non-pathogenic antigens, and are the primary modulators of peripheral tolerance. Vignali et al., How regulatory T cells work. Nat. Rev. Immunol. 2008 July; 8(7): pp. 523-32. Treg cells can be tuned to tolerate select antigens through exposure to these stimuli in vivo or ex vivo. Studies have suggested that Treg cells can be used to prevent or treat transplant rejection. However, clinical implementation of Treg cell therapy is hindered by multiple factors, including the need to identify donor-specific antigens before transplantation, a prerequisite that is often not feasible in clinical settings, especially due to the limited time period between identification of the donor-recipient organ allocation and the transplant. With a deceased donor, potent Tregs with broad specificity to a spectrum of donor antigens are required if they are to be administered at the time of transplant.

Polyclonal Treg cells can be generated by non-specific T-cell receptor stimulation. Singer et al., Regulatory T cells as immunotherapy. Front Immunol. 2014, 5:46. For example, polyclonal Tregs can be generated by expanding sorted natural Tregs using anti-CD3 antibodies, anti-CD28 antibodies, IL-2 and “artificial APCs” to nonspecifically expand Tregs. Although polyclonal Tregs generated by existing methods are being used in clinical trials in autoimmune disease and transplantation trials, these Tregs are not very stable in their function or phenotype and do not have optimal effects in vivo.

Treg cells can also be generated specifically by exposure to selected antigen presenting cells (APCs). Donor-specific Tregs can be generated by using sBcs from the transplant donor. However, with a living donor, these Tregs must be generated prior to the transplantation.

B cells exert their influence on the immune system through the production of antibodies, antigen presentation and cytokine production. Wang et al., Regulatory T cells and B cells: implication on autoimmune diseases. Intl. J. Clin. Exp. Pathol. 2013, 6(12):2668-74.

U.S. Patent Publication No. 20150110761 describes using the CD40-stimulated B cells from the donor to generate donor-specific Treg cells, not polyclonal Treg cells. This method to generate donor-specific Treg cells is not suitable for Treg administration in a transplantation with a deceased donor. While Landwehr-Kenzel et al. discuss a bank of B cells, it is still in the context of donor-specific Treg expansion, not polyclonal Tregs. Landwehr-Kenzel et al., Novel GMP-Compatible Protocol Employing an Allogeneic B Cell Bank for Clonal Expansion of Allospecific Natural Regulatory T Cells, American Journal of Transplantation 2014; 14: 594-606.

### SUMMARY OF THE INVENTION

In certain embodiments, the present invention relates to a method of producing polyclonal regulatory T (Treg) cells, the method comprising: co-culturing (i) stimulated B cells (sBcs) prepared from a plurality of human leukocyte antigen (HLA)-typed donors with (ii) CD4+, CD25+/hi and CD127−/lo T cells from a recipient to generate polyclonal Treg cells.

In certain embodiments, CD25hi=top 5% of CD25 expression; CD12710=bottom 5% of the CD127-expressing cells.

In certain embodiments, the sBcs are prepared by co-culturing CD154-expressing cells with a pool of peripheral blood mononuclear cells (PBMCs) from the plurality of HLA-typed donors.

In additional embodiments, the CD154-expressing cells are fibroblast cells.

In further embodiments, the sBcs are irradiated prior to co-culturing with the T cells.

In additional embodiments, in the co-culturing step, the ratio of the T cells to the sBcs ranges from about 1:50 to about 4:1. In additional embodiments, the ratio of the T cells to the sBcs is about 1:4. In additional embodiments, the T cells and the sBcs are co-cultured in a medium comprising interleukin-2 (IL-2).

In additional embodiments, the polyclonal Tregs are re-stimulated by co-culturing with a second set of sBcs prepared from a plurality of human leukocyte antigen (HLA)-typed donors.

In additional embodiments, the T cells are isolated from peripheral blood mononuclear cells (PBMCs) of the recipient.

In certain embodiments, the present invention relates to a composition comprising a population of the Treg cells produced by any of the methods described herein.

In certain embodiments, the present invention relates to a method for treating or preventing rejection of an allograft in a recipient, the method comprising: administering the composition as described herein/above, to the recipient before, during or after a transplant of the allograft.

In certain embodiments, the allograft is a solid organ allograft.

In certain embodiments, the solid organ allograft is selected from the group consisting of cardiac, lung, cardiac/lung, kidney, pancreas, kidney/pancreas, liver, intestine, and skin allografts.

In certain embodiments, the composition is administered to the recipient concurrently with prednisone, mycophenolate mofetil, sirolimus and/or tacrolimus.

In certain embodiments, the present invention relates to a method of treating or preventing an autoimmune disease in a subject, the method comprising: administering the composition described herein/above, to the subject.

In certain embodiments, the present invention relates to a method of producing polyclonal regulatory T (Treg) cells, the method comprising:
(a) generating stimulated B cells (sBcs) from a plurality of human leukocyte antigen (HLA)-typed donors,
(b) isolating CD4+, CD25+/hi and CD127−/lo T cells from a recipient, and
(c) co-culturing the sBcs of step (a) with the T cells of step (b) to produce polyclonal Treg cells.

In additional embodiments, the sBcs are generated by co-culturing CD154-expressing cells with a pool of peripheral blood mononuclear cells (PBMCs) from the plurality of HLA-typed donors.

In additional embodiments, the CD154-expressing cells are fibroblast cells.

In additional embodiments, the sBcs are irradiated prior to co-culturing with the T cells.

In additional embodiments, in step (c) the ratio of the T cells to the sBcs ranges from about 1:50 to about 4:1.

In additional embodiments, in step (c) the ratio of the T cells to the sBcs is about 1:4.

In additional embodiments, the T cells and the sBcs are co-cultured in a medium comprising interleukin-2 (IL-2).

In certain embodiments, the present invention relates to a kit comprising the composition described herein/above.

In additional embodiments, the kit further comprises one or more additional components selected from the group consisting of culture media, buffers, growth factors, and additional cell lines.

In certain embodiments, the sBc donor pool will be selected such that, >50% of organ donors at the center express at least one HLA represented in the sBc donor pool. In certain embodiments, the donor pool will consist of sBcs from 4-20 donors.

### BRIEF DESCRIPTION OF THE DRAWINGS

FIG. 1 is a graph illustrating donor chimerism following bone marrow transplant with infusion of Tregs generated by the protocols/methods described herein in Examples 1-3. Shown is the percent of peripheral blood cells that arise from the bone marrow donor (chimerism) after bone marrow transplant and Treg infusion. The recipient developed full donor chimerism of multiple hematopoietic cell lineages.

### DETAILED DESCRIPTION

The present disclosure provides for methods to produce B cell-expanded Tregs that are more stable and more effective than donor-specific Tregs for suppressing polyclonally activated T cells. In certain embodiments, the present polyclonal Tregs can react to many donors, not a specific donor.

The polyclonal Tregs generated by the present methods can be administered before, during or after a transplantation (e.g., on the day of transplantation), such as with a deceased donor.

The Tregs generated by the present method are polyclonal with reactivity against a broad array of potential organ donors. In certain embodiments, the present method obtains a collection of HLA-typed blood cells from healthy donors with which to create genetically diverse B cell pools that would be used to stimulate recipient Treg expansion. The present methods generate potent polyclonal Tregs for treating or preventing transplant rejection, e.g., involving a deceased donor.

The present Treg cells can also prevent or treat graft-versus-host disease (e.g., in hematopoietic cell transplantation), or autoimmune diseases. The present disclosure also provides for methods of generating banks of various Treg cells for personalized medicine. The present disclosure provides for methods to prevent or treat allergies.

The present disclosure provides for an ex vivo method to generate polyclonal Treg cells that tolerate a broad spectrum of antigens with high prevalence in the general population. The present Treg cells can be used to suppress or prevent transplant rejection (e.g., in solid organ transplantation) where the specific antigens in donor organs, tissues or cells are unknown until just prior to transplantation (e.g., with a deceased donor) with minimal processing time. The present method allows for the timely administration of immune-tolerant Treg cells that prevent or treat transplant rejection.

In certain embodiments, the present method generates high-quality Tregs that can be administered to a transplant recipient receiving an allograft from a deceased donor where the donor HLA is not known until a short time period (e.g., a few hours) prior to the transplant, precluding the generation of Tregs with specific activity against the donor.

In certain embodiments, polyclonal Treg cells are generated by co-culturing natural Treg cells isolated from a recipient with a pool of donor-derived, stimulated B cells (sBcs) that express antigens with a high prevalence in the general population.

The sBcs may be cryopreserved prior to co-culturing with natural Treg cells, or may be prepared freshly before co-culturing with natural Treg cells.

In certain embodiments, when natural Treg cells to sBcs are co-cultured, the ratio of natural Treg cells to sBcs may range from about 1:100 to about 20:1, about 1:80 to about 15:1, about 1:60 to about 10:1, about 1:50 to about 10:1, about 1:50 to about 8:1, about 1:50 to about 6:1, about 1:50 to about 4:1, about 1:40 to about 10:1, about 1:40 to about 8:1, about 1:40 to about 6:1, about 1:40 to about 4:1, about 1:30 to about 10:1, about 1:30 to about 8:1, about 1:30 to about 6:1, about 1:30 to about 4:1, about 1:20 to about 5:1, about 1:20 to about 3:1, about 1:20 to about 2:1, about 1:20 to about 1:1, about 1:10 to about 5:1, about 1:10 to about 4:1, about 1:10 to about 3:1, about 1:10 to about 2:1, about 1:10 to about 1:1, about 1:5 to about 2:1, about 1:5 to about 1:1, about 1:4 to about 2:1, or about 1:4 to about 1:1. In certain embodiments, the ratio of natural Treg cells to sBcs is about 1:10. In certain embodiments, the ratio of natural Treg cells to sBcs is about 1:4.

In certain embodiments, blood from a plurality of donors is drawn and pooled, where the plurality of donors express human leukocyte antigens (HLA) with high prevalence in the general population. Peripheral blood mononuclear cells (PBMCs) are isolated from the pooled blood, and then cultured with fibroblast cells that express CD154 (CD40 ligand or CD40L) to generate stimulated B cells (sBcs) that express high levels of the T-cell co-stimulatory marker CD40.

Discriminatory classification of HLA allelic variation on the basis of observed population allele frequencies (common, rare and unseen) for HLA-A, -C, -B, -DRB1, -DQA1, and -DQB1 can be found at https://bioinformatics.bethematchclinical.org/hla-resources/haplotype-frequencies/high-resolution-hla-alleles-and-halotypes-in-the-us-population/. High resolution HLA alleles and haplotypes in the US population. Human Immunology (2007) 68, 779-788. The makeup of the donor pool will vary depending on the specific HLA prevalence at the particular transplant center. In certain embodiments, the sBc donor pool will be selected such that, >50% of organ donors at the center express at least one HLA represented in the sBc donor pool. In certain embodiments, the donor pool will consist of sBcs from 4-20 donors.

In certain embodiments, natural Treg cells are isolated from the blood of a transplant recipient by, e.g., fluorescence-activated cell sorting (FACS) for CD4+/CD25hi/CD127− cells. In certain embodiments, natural Treg cells are T cells that are CD4+, CD25+/hi and CD127−/lo. For reference, it is noted that CD25hi=top 5% of CD25 expression; CD12710=bottom 5% of the CD127-expressing cells. In certain embodiments, Tregs express the biomarkers CD4, FOXP3, and CD25.

The sBcs may or may not be irradiated prior to co-culturing with the natural Treg cells.

The isolated natural Treg cells are then incubated with the irradiated (or non-irradiated) sBcs to generate polyclonal Tregs with reactivity to antigens that are highly prevalent in the general population.

In certain embodiments, blood is drawn from donors that express human leukocyte antigens (HLA) with high prevalence in the general population. Peripheral blood mononuclear cells (PBMC) are isolated with density gradient centrifugation. The PBMC are then cultured with fibroblast cells (e.g., 3T3 cells) that expresses CD154 (CD40 ligand or CD40L). This generates stimulated B cells (sBcs) that express high levels of the T-cell costimulatory marker CD40. Blood is drawn from a putative transplant recipient and PBMCs are isolated, e.g., by density gradient centrifugation. Tregs are isolated from the PBMCs by fluorescence-activated cell sorting (FACS) to obtain the CD4+/CD25hi/CD127− fraction. The Tregs are then incubated with the irradiated sBc cells from a pool of donors to generate polyclonal Tregs with reactivity to antigens with high prevalence in the general population.

### Definitions

“Transplantation” refers to the process of transferring (moving) a transplantable graft into a recipient subject (e.g., from a donor subject, from an in vitro source (e.g., differentiated autologous or heterologous native or induced pluripotent cells)) and/or from one bodily location to another bodily location in the same subject.

“Undesired immune response” refers to any undesired immune response that results from exposure to an antigen, promotes or exacerbates a disease, disorder or condition provided herein (or a symptom thereof), or is symptomatic of a disease, disorder or condition provided herein. Such immune responses generally have a negative impact on a subject's health or is symptomatic of a negative impact on a subject's health.

In an embodiment, the transplanted tissue is lung tissue, heart tissue, kidney tissue, liver tissue, retinal tissue, corneal tissue, skin tissue, pancreatic tissue, intestinal tissue, genital tissue, ovary tissue, bone tissue, tendon tissue, or vascular tissue.

As used herein a “recipient subject” is a subject who is to receive, or who has received, a transplanted cell, tissue or organ from another subject.

As used herein a “donor subject” is a subject from whom a cell, tissue or organ to be transplanted is removed before transplantation of that cell, tissue or organ to a recipient subject.

A “transplantable graft” refers to a biological material, such as cells, tissues and organs (in whole or in part) that can be administered to a subject. Transplantable grafts may be autografts, allografts, or xenografts of, for example, a biological material such as an organ, tissue, skin, bone, nerves, tendon, neurons, blood vessels, fat, cornea, pluripotent cells, differentiated cells (obtained or derived in vivo or in vitro), etc. In some embodiments, a transplantable graft is formed, for example, from cartilage, bone, extracellular matrix, or collagen matrices. Transplantable grafts may also be single cells, suspensions of cells and cells in tissues and organs that can be transplanted. Transplantable cells typically have a therapeutic function, for example, a function that is lacking or diminished in a recipient subject. Some non-limiting examples of transplantable cells are islet cells, β-cells, hepatocytes, hematopoietic stem cells, neuronal stem cells, neurons, glial cells, or myelinating cells. Transplantable cells can be cells that are unmodified, for example, cells obtained from a donor subject and usable in transplantation without any genetic or epigenetic modifications. In other embodiments, transplantable cells can be modified cells, for example, cells obtained from a subject having a genetic defect, in which the genetic defect has been corrected, or cells that are derived from reprogrammed cells, for example, differentiated cells derived from cells obtained from a subject.

In an embodiment the donor subject is a human. In an embodiment the recipient subject is a human. In an embodiment both the donor and recipient subjects are human. As used herein “rejection by an immune system” describes the event of hyperacute, acute and/or chronic response of a recipient subject's immune system recognizing a transplanted cell, tissue or organ from a donor as non-self and the consequent immune response.

The term “allogeneic” refers to any material derived from a different animal of the same species as the individual to whom the material is introduced. Two or more individuals are said to be allogeneic to one another when the genes at one or more loci are not identical.

The term “autologous” refers to any material derived from the same individual to whom it is later to be re-introduced into the same individual.

As used herein, a “prophylactically effective” amount is an amount of a substance effective to prevent or to delay the onset of a given pathological condition in a subject to which the substance is to be administered. A prophylactically effective amount refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

As used herein, a “therapeutically effective” amount is an amount of a substance effective to treat, ameliorate or lessen a symptom or cause of a given pathological condition in a subject suffering therefrom to which the substance is to be administered.

“Treating” or “treatment” of a state, disorder or condition includes:
(1) preventing or delaying the appearance of clinical symptoms of the state, disorder, or condition developing in a person who may be afflicted with or predisposed to the state, disorder or condition but does not yet experience or display clinical symptoms of the state, disorder or condition; or
(2) inhibiting the state, disorder or condition, i.e., arresting, reducing or delaying the development of the disease or a relapse thereof (in case of maintenance treatment) or at least one clinical symptom, sign, or test, thereof; or
(3) relieving the disease, i.e., causing regression of the state, disorder or condition or at least one of its clinical or sub-clinical symptoms or signs.

The benefit to a subject to be treated is either statistically significant or at least perceptible to the patient or to the physician.

“Activation,” “stimulation,” and “treatment,” as it applies to cells or to receptors, may have the same meaning, e.g., activation, stimulation, or treatment of a cell or receptor with a ligand, unless indicated otherwise by the context or explicitly. “Ligand” encompasses natural and synthetic ligands, e.g., cytokines, cytokine variants, analogues, muteins, and binding compounds derived from antibodies. “Ligand” also encompasses small molecules, e.g., peptide mimetics of cytokines and peptide mimetics of antibodies. “Activation” can refer to cell activation as regulated by internal mechanisms as well as by external or environmental factors. “Response,” e.g., of a cell, tissue, organ, or organism, encompasses a change in biochemical or physiological behavior, e.g., concentration, density, adhesion, or migration within a biological compartment, rate of gene expression, or state of differentiation, where the change is correlated with activation, stimulation, or treatment, or with internal mechanisms such as genetic programming.

“Activity” of a molecule may describe or refer to the binding of the molecule to a ligand or to a receptor, to catalytic activity; to the ability to stimulate gene expression or cell signaling, differentiation, or maturation; to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” can also mean specific activity, e.g., [catalytic activity]/[mg protein], or [immunological activity]/[mg protein], concentration in a biological compartment, or the like. “Activity” may refer to modulation of components of the innate or the adaptive immune systems.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., rat, mouse, dog, cat, rabbit) and most preferably a human.

“Treat” or “treating” means to administer a therapeutic agent, such as a composition containing any compound or therapeutic agent of the present invention, internally or externally to a subject or patient having one or more disease symptoms, or being suspected of having a disease or being at elevated at risk of acquiring a disease, for which the agent has therapeutic activity. Typically, the agent is administered in an amount effective to alleviate one or more disease symptoms in the treated subject or population, whether by inducing the regression of or inhibiting the progression of such symptom(s) by any clinically measurable degree. The amount of a therapeutic agent that is effective to alleviate any particular disease symptom (also referred to as the “therapeutically effective amount”) may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom.

In an embodiment of the invention, a composition of the invention is administered to a subject in accordance with the Physicians' Desk Reference 2003 (Thomson Healthcare; 57th edition (Nov. 1, 2002)).

Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopoeia or other generally recognized pharmacopeias for use in animals, and more particularly in humans.

“Patient” or “subject” refers to mammals and includes human and veterinary subjects.

### Kits

The present invention also provides kits comprising the components of the combinations of the disclosure in kit form. A kit may include one or more components including, but not limited to, any of the therapeutic compositions or cellular components or cell lines, as discussed herein, optionally in association with one or more additional components including, a therapeutic agent, as discussed herein. The compositions and/or the therapeutic agent/s can be formulated as a pure composition or in combination with a pharmaceutically acceptable carrier, in a pharmaceutical composition.

In one embodiment, a kit includes the any of the therapeutic compositions or cellular components, each in a separate container (e.g., in a sterile glass or plastic vial). The kit can include a package insert including information concerning cell growth and maintenance, as well as buffers and/or growth factors in the kit.

To prepare pharmaceutical or sterile compositions of the compositions of the present invention, the compounds or cells, or similar compositions may be admixed with a pharmaceutically acceptable carrier or excipient. See, e.g., Remington's Pharmaceutical Sciences and U.S. Pharmacopeia: National Formulary, Mack Publishing Company, Easton, Pa. (1984).

Formulations of therapeutic and diagnostic agents may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

The following are examples of the present disclosure and are not to be construed as limiting.

### Example 1 sBc Expansion and Maintenance of 3T3-CD40L Cells

Prepare cultures or continuation lines of 3T3-CD40L cells several days prior.

### 1. Prepare PBMCs.

### 2. Prepare sBc Culture Medium:

450 mL of X-Vivo 15
50 mL of Human AB Serum
0.724 mL regular insulin, 100 U/ml
   3. Prepare 3T3 growth medium and 3T3 wash medium
445 mL IMDM (485 mL for wash medium)
50 mL FCS (2% FCS for wash medium, 10 mL)
5 mL Penicillin/streptomycin.
0.5 mL Cipro

### Preparation of 3T3-CD40L Cells:

Thawing 3T3 Cells from frozen vial
1. Fill one T75 flask with 40 ml of 3T3 Medium.
2. Thaw one vial of frozen 3T3 cells.
3. Transfer the cells to the T75 flask. Place the flask in a 37C/5% CO2 incubator for 3-4 hours.
4. Remove the medium from the T75 (by this time the cells should be adherent to the bottom of the flask). Add 50 ml of fresh medium to the flask and return to the incubator. Wait until the following day to use the cells, or leave for 2-3 days to initiate a continuation line.

### Continuation and Harvesting of 3T3 Cells:

1. Empty the T75 flask containing 3T3 cells by pipetting off the supernatant.
2. Add 5 ml of PBS to rinse the flask.
3. Add 1-3 ml 0.05% Trypsin-EDTA to the flask, briefly swirl flask to coat and pipette off Trypsin.
4. Add 4-6 ml 0.05% Trypsin-EDTA to the flask and incubate for 2-4 min
5. Tap the sides of the flask gently to see if cells are being released from the bottom of the flask.
6. Add 9-10 ml of 3T3 Wash Media and pipet up and down to mix. Harvest the contents of the flask into a 50-ml conical tube.
7. Spin down at 1500 rpm for 5 min. at RT.
8. Aspirate the supernatant and resuspend the pellet by tapping the side of the tube.
9. Add 5 ml of 3T3 Growth medium to the tube. Count the cells.
10. Fill a new T25 flask with 8 ml or a T75 flask with 40 ml of 3T3 Medium (depending on how many 3T3 cells that will be needed). Transfer 0.25×10⁶ cells to the T25 or 0.5×10⁶ cells (or 50,000 cells) to the T75 and place in the incubator, if not needed for 7 days.
11. The remaining 3T3-CD40L cells will be irradiated for stimulating B cells. Irradiate the 3T3 cells for 46 gray.
   Stimulated B Cell (sBc) Culture:

### Day 0—Initiation of Culture:

Prepare 3T3-CD40L cells at 500,000 cells per ml in sBc culture medium.
PBMCs are prepared in sBc culture medium, at 125,000 B cells per ml (e.g., assuming 10% of PBMC are B-cells)
Add the following to the PBMCs in sBc culture medium: 1 μl IL-4 (stock 8 μg/ml), 2 μl CSA (stock 1 mg/ml), and 20 μl GCV (stock 0.5 mg/ml) per ml of sBc culture medium so that the final concentration when the 3T3-CD40L cells are added is 4 ng/ml IL-4, 1 μg/ml CSA, and 5 μg/ml GCV. Mix well by gentle swirling.
Transfer 1.0 ml of PBMC cell suspension to each well of a 6-well plate.
Add 1 ml of 3T3-CD40L cells to each well containing PBMCs.
There should be 2 ml/well.
Transfer plate to 37° C./5% CO2 incubator.

### Day 2—Feeding: Doubling of Medium:

Remove cell culture vessel from incubator and visualize cells using microscope. B cells should begin to adhere to the 3T3-CD40L cells.
Add 2 ml of fresh sBc culture medium containing 0.5 μl IL-4 (stock 8 μg/ml), 1 μl CSA (stock 1 μg/ml), and 10 μl GCV (stock 0.5 mg/ml) per ml to each well so that the final concentration is 4 ng/ml IL4, 1 mg/ml CSA, and 5 μg/ml GCV.
Transfer plate to 37° C./5% CO2 incubator.

### Day 4—Feeding: Doubling of Medium:

Remove cell culture vessel from incubator and visualize cells using microscope.
Add 4 ml of sBc culture medium per well that has been supplemented with 0.5 μl IL-4 (stock 8 μg/ml), 1 μl CSA (stock 1 mg/ml), and 10 μl GCV (stock 0.5 mg/ml) per ml to each well so that the final concentration is 4 ng/ml IL4, 1 μg/ml CSA, and 5 μg/ml GCV.
Transfer plate to 37° C./5% CO2 incubator.
   Day 7—Re-stimulation of sBc with CD40L cells:
Adjust CD40L cells to 100,000-500,000 cells/ml in sBc culture medium.
Harvest the sBcs into a conical tube.
Centrifuge cells at 470×g for 5 minutes at 20° C.
Discard remaining supernatant from pellet and resuspend the pellet in sBc culture medium. Count cells.
Dilute sBc to a density of 1×10⁶ cells/ml in sBc culture medium.
Add 0.5 μl IL-4 (stock 8 μg/ml) and 10 μl GCV (stock 0.5 mg/ml) per ml sBc culture medium so that the final concentration is 4 ng/ml IL4 and 5 μg/ml GCV. Mix by gently swirling.
Transfer sBc to the wells or flasks with appropriate volume of irradiated CD40L cells and additional sBc culture medium (Table 1).
Transfer plate to 37° C./5% CO2 incubator.

TABLE 1

| | | CD40L | sBc culture | Total |
|---|---|---|---|---|
| Plate/ | sBc | Cells | medium | Volume |
| Flask | (ml) | (mL) | (ml) | (mL) |
| 6 well | 1 | 1 | 2 | 4 |
| T25 | 3-6 | 3-6 | 6-12 | 12-24 |
| T75 | 8-14 | 8-14 | 16-28 | 32-56 |
| T150 | 16-20 | 16-20 | 32-40 | 64-80 |

### Day 10—sBc Harvest:

Remove cell culture vessel(s) from incubator and visualize cells using microscope.
Harvest the sBcs into one container.
Centrifuge cells at 470×g for 5 minutes at 20° C.
Discard remaining supernatant from pellet and resuspend the pellet in ˜5-10 ml of sBc culture medium in a 50 ml conical tube. Count cells.
   If starting a Treg culture on same day:
Determine the number of sBc to be irradiated and set aside appropriate amount of cells into a new 50 ml conical tube.
   If NOT starting a Treg culture on the same day:
Freeze cells for later use (see Cell Freezing protocol)
   Pooled sBc from multiple donors are used to generate polyclonal Tregs (see Example 2)

### REFERENCES

Adapted from: Putnam et al., Expansion of Human Regulatory T Cells from Patients with Type 1 Diabetes, Diabetes, 2009 March; 58(3):652-62.

### Example 2 Polyclonal Treg Culture

### Reagents

Treg culture media (500 mL) sterile filtered
450 mL X-Vivo 15 (light sensitive)
50 mL decomplemented human serum
Human IL-2: stock solution: 50,000 IU/mL in PBS

Table 2 determines the plating scheme based on the number of Tregs.

TABLE 2

| # of Tregs | Vessel | Media Volume |
|---|---|---|
| 100,000-150,000 | 48 well | 1 mL |
| 200,000-300,000 | 24 well | 2 mL |
| 400,000 | 12 well | 3 mL |
| 800,000 | 6 well | 4 mL |
| 1,200,000 | Vertical T25 | 6 mL |
| 2,400,000 | Horizontal T25 | 10 mL |
| 7,500,000 | T75 | 30 mL |
| 17,000,000 | T175 | 70 mL |

### Procedures:

sBc culture must be performed prior to this protocol.
Day −1 (afternoon/evening)—preparation for Treg sort
1. Thaw PBMCs into MLR media, spin, wash with MLR, and count in MLR. (N.B. MACS no touch T cell isolation can be performed at this step.)
2. Resuspend PBMCs at 2-4×10⁶ per mL in MLR, and add IL-2 such that the IL-2 concentration is 200 IU/mL (4 uL of stock IL-2 solution per mL of cell suspension).
3. Rest cells in 6 well plate (8 mL of cell suspension per well), 12 well plate (3 mL per well), etc.
   Day 0 (morning)—Treg sort and setup of polyclonal Treg culture
1. Harvest cells from overnight culture, spin, and count.
2. Stain cells for Treg sort: CD4—AF700, CD25—PE, CD127—BV421
3. Sort into 5 mL of Treg culture media
4. Setup Treg culture:
   a. Following sort, spin cells down, resuspend in 1 mL Treg culture media, and count.
   b. Add irradiated (30 Gy) sBcs from pooled donors (either thawed or freshly made) to Tregs such that the ratio is 1 Treg: 4 sBc.
   c. Add IL-2 such that final concentration of IL-2 is 300 IU/mL (6 uL of stock IL-2 solution per mL of cell suspension)
   d. Plate Tregs into wells based on Table 2.
      Days 2, 5, 7, 9—Feeding/changing media
1. View cells under microscope; there should be large clumps, even on day 2.
2. Harvest polyclonal Treg culture from plate and spin at 1500, 5 min, RT.
3. Resuspend in Treg culture media and count cells.
4. Re-plate cells according to Table 2, with proper volume of Treg culture media and a final concentration of 300 IU/mL of IL-2 (6 uL of stock IL-2 solution per mL of cell suspension).
   Day 11—re-stimulation of culture
1. Have irradiated pooled donor sBcs ready (30 Gy) for restimulation.
2. View cells under microscope; there should be large clumps.
3. Harvest polyclonal Treg culture from plate and spin at 1500, 5 min, RT.
4. Resuspend in Treg culture media and count cells.
5. Make cell suspension of 1 Treg: 4 sBc (newly irradiated) with 300 IU/mL of IL-2 (6 uL of stock IL-2 solution per mL of cell suspension) and re-plate according to Table 2.
   Day 14—Feeding/changing media
1. View cells under microscope; there should be large clumps.
2. Harvest polyclonal Treg culture from plate and spin at 1500, 5 min, RT.
3. Resuspend in Treg culture media and count cells.
4. Re-plate cells according to table at beginning of protocol, with proper volume of Treg culture media and a final concentration of 300 IU/mL of IL-2 (6 μL of stock IL-2 solution per mL of cell suspension).
   Day 16—Take down
1. View cells under microscope; there should be large clumps.
2. Harvest polyclonal Treg culture from plate and spin at 1500, 5 min, RT.
3. Resuspend and count cells.
4. Cryopreserve cells until date of infusion.

### Example 3

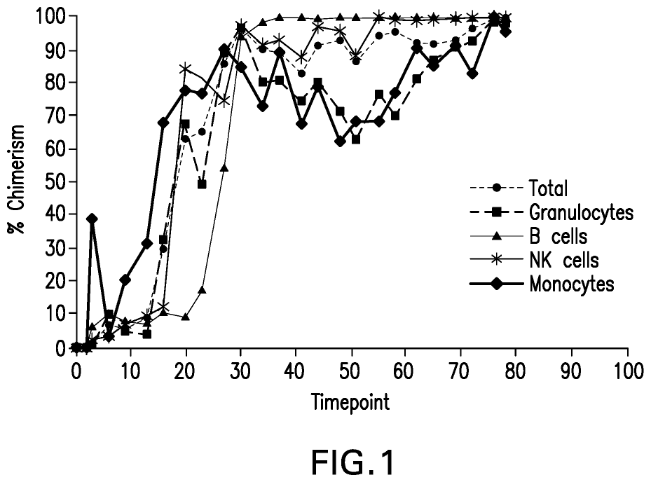

The first animal to receive Tregs generated in accordance with the present methods, was transplanted on 9/7/16, with a total of 3 animals having received Tregs generated by these methods/protocols as of June, 2018. The tolerance induction regimen included total body irradiation (125 cGy) on POD −6 and POD −5, thymic irradiation (700 cGy) on POD −1, ATGAM (50 mg/kg) on POD −2, −1, and 0, anti-CD154 mAb (clone 5c8, 25 mg/kg) on POD 0, 2, 5, 7, 9, and 12 and rapamycin IM from POD 0-30. The animals received donor bone marrow transplantation on POD 2 with Treg infusions on POD 2, 4, 7, 9 and 55. Two of the three animals that received Tregs generated with this invention developed full donor chimerism in multiple hematopoietic lineages (FIG. 1). FIG. 1 is a graph illustrating donor chimerism following bone marrow transplant with infusion of Tregs generated by the protocols/methods described herein in Examples 1-3. Shown is the percent of peripheral blood cells that arise from the bone marrow donor (chimerism) after bone marrow transplant and Treg infusion. The recipient developed full donor chimerism of multiple hematopoietic cell lineages.

Thus, to summarize, regulatory T (Treg) cells have demonstrated the potential to prevent transplant rejection. However, Treg cells with broad specificity to a spectrum of donor antigens must be generated and administered at the time of transplant, which is frequently challenging due to the short time between identification of the donor-recipient organ allocation and surgery. The present technology in certain embodiments, provides methods to generate Treg cells that tolerate a broad spectrum of antigens with high prevalence in the general population. These methods can be used to generate Treg cells that can be used to suppress rejection responses to donor organs where the specific antigens are unknown with minimal processing time. Additionally, the methods described herein can also be adapted to generate Treg cells for prevention of graft vs. host disease following hematopoietic cell transplantation, or for treatment of autoimmune diseases.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description is offered by way of illustration only and not as a limitation.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

1. A method of producing polyclonal regulatory T (Treg) cells, the method comprising:
co-culturing (i) stimulated B cells (sBcs) prepared from a plurality of human leukocyte antigen (HLA)-typed donors with (ii) CD4+, CD25+/hi and CD127−/lo T cells from a recipient to generate polyclonal Treg cells.

2. The method of claim 1, wherein the sBcs are prepared by co-culturing CD154-expressing cells with a pool of peripheral blood mononuclear cells (PBMCs) from the plurality of HLA-typed donors.

3. The method of claim 2, wherein the CD154-expressing cells are fibroblast cells.

4. The method of claim 1, wherein the sBcs are irradiated prior to co-culturing with the T cells.

5. The method of claim 1, wherein in the co-culturing step the ratio of the T cells to the sBcs ranges from about 1:50 to about 4:1.

6. (canceled)

7. The method of claim 1, wherein the T cells and the sBcs are co-cultured in a medium comprising interleukin-2 (IL-2).

8. The method of claim 1, wherein the polyclonal Tregs are re-stimulated by co-culturing with a second set of sBcs prepared from a plurality of human leukocyte antigen (HLA)-typed donors.

9. The method of claim 1, wherein the T cells are isolated from peripheral blood mononuclear cells (PBMCs) of the recipient.

10. A composition comprising a population of the Treg cells produced by the method of claim 1.

11. A method for treating or preventing rejection of an allograft in a recipient, the method comprising: administering the composition of claim 10 to the recipient before, during or after a transplant of the allograft.

12. The method of claim 11, wherein the allograft is a solid organ allograft.

13. The method of claim 12, wherein the solid organ allograft is selected from the group consisting of cardiac, lung, cardiac/lung, kidney, pancreas, kidney/pancreas, liver, intestine, and skin allografts.

14. The method of claim 11, wherein the composition is administered to the recipient concurrently with prednisone, mycophenolate mofetil, sirolimus and/or tacrolimus.

15. A method of treating or preventing an autoimmune disease in a subject, the method comprising: administering the composition of claim 10 to the subject.

16. A method of producing polyclonal regulatory T (Treg) cells, the method comprising:
(a) generating stimulated B cells (sBcs) from a plurality of human leukocyte antigen (HLA)-typed donors,
(b) isolating CD4+, CD25+/hi and CD127−/lo T cells from a recipient, and
(c) co-culturing the sBcs of step (a) with the T cells of step (b) to produce polyclonal Treg cells.

17. The method of claim 16, wherein the sBcs are generated by co-culturing CD154-expressing cells with a pool of peripheral blood mononuclear cells (PBMCs) from the plurality of HLA-typed donors.

18. The method of claim 17, wherein the CD154-expressing cells are fibroblast cells.

19. The method of claim 16, wherein the sBcs are irradiated prior to co-culturing with the T cells.

20. The method of claim 16, wherein in step (c) the ratio of the T cells to the sBcs ranges from about 1:50 to about 4:1.

21. (canceled)

22. The method of claim 16, wherein the T cells and the sBcs are co-cultured in a medium comprising interleukin-2 (IL-2).

23. (canceled)

24. (canceled)
